(19) United States
(12) Patent Application Publication
(10) Pub. No.: US 2023/0212105 A1
(43) Pub. Date: Jul. 6, 2023

(54) FORMING ACETIC ACID BY THE SELECTIVE OXIDATION OF METHANE

(71) Applicant: NOVA CHEMICALS (INTERNATIONAL) S.A., Fribourg (CH)

(72) Inventors: Shahin GOODARZNIA, Calgary (CA); Bolaji OLAYIWOLA, Calgary (CA); Vasily SIMANZHENKOV, Calgary (CA); Aashish GAURAV, Calgary (CA)

(21) Appl. No.: 18/008,783

(22) PCT Filed: May 25, 2021
(86) PCT No.: PCT/IB2021/054552

§ 371 Date: Dec. 7, 2022

Related U.S. Application Data
(60) Provisional application No. 63/036,476, filed on Jun. 9, 2020.

(51) Int. Cl. C07C 51/16; C07C 51/42; C07C 51/15; B01J 23/652; B01J 8/06

(52) U.S. Cl. CPC C07C 51/16; C07C 51/42; C07C 51/15; B01J 23/6525; B01J 8/065; B01J 2208/02; B01J 2208/065; B01J 2208/00053; B01J 2208/00212

(57) ABSTRACT

Methods and a reactor system for producing acetic acid in a selective oxidation (SO) reactor are provided. An example method includes providing a fresh feed stream to the SO reactor, wherein the fresh feed stream includes a methane feed stream, a carbon dioxide feed stream, and a steam feed stream. Acetic acid is formed in the SO reactor. An acetic acid product stream is separated from a reactor effluent stream in a scrubber. A recycle gas stream is obtained from the scrubber. At least a portion of the recycle gas stream is combined into the fresh feed stream to the SO reactor.

### TECHNICAL FIELD

The present disclosure relates generally to selective oxidation (SO) of lower alkanes to form acetic acid in large amounts.

### BACKGROUND ART

Olefins like ethylene, propylene, and butylene, are basic building blocks for a variety of commercially valuable polymers. Since naturally occurring sources of olefins do not exist in commercial quantities, polymer producers rely on methods for converting the more abundant lower alkanes into olefins. The method of choice for today's commercial scale producers is steam cracking, a highly endothermic process where steam-diluted alkanes are subjected very briefly to a temperature of at least 800° C. The fuel demand to produce the required temperatures and the need for equipment that can withstand that temperature add significantly to the overall cost. Also, the high temperature promotes the formation of coke which accumulates within the system, resulting in the need for costly periodic reactor shut-down for maintenance and coke removal.

Selective oxidation processes, such as oxidative dehydrogenation (ODH), are an alternative to steam cracking that are exothermic and produce little or no coke. In ODH, a lower alkane, such as ethane, is mixed with oxygen in the presence of a catalyst and optionally an inert diluent, such as carbon dioxide or nitrogen or steam, in some embodiments at temperatures as low as 300° C., to produce the corresponding alkene. In some embodiments, various other oxidation products may also be produced in this process.

### SUMMARY OF INVENTION

An embodiment described herein provides a method for producing acetic acid in a selective oxidation (SO) reactor. The method includes providing a fresh feed stream to the SO reactor. The fresh feed stream includes a methane feed stream, a carbon dioxide feed stream, and a steam feed stream. Acetic acid is formed in the SO reactor. An acetic acid product stream is separated from a reactor effluent stream in a scrubber. A recycle gas stream is obtained from the scrubber, and at least a portion of the recycle gas stream is combined into the fresh feed stream to the SO reactor. In an aspect, the recycle gas stream includes methane.

In an aspect, adding the fresh feed stream to the SO reactor includes adding two or more feed streams separately to the SO reactor. An amount of the steam feed stream added to the SO reactor may be adjusted to increase a selectivity for acetic acid.

In an aspect, a flue gas stream from a combustion process is used as the carbon dioxide feed stream. In an aspect, a natural gas stream from a pipeline is used as the methane feed stream. In an aspect, the method includes separating a recycled water stream from the acetic acid and combining at least a portion of the recycled water stream with the steam feed stream.

In an aspect, forming the acetic acid in the SO reactor includes placing the fresh feed stream in contact with an SO catalyst of the formula:

MOₐV_{b}Te_{c}Nb_{d}PdₑO_{f}

wherein a, b, c, d, e, and f are relative atomic amounts of the elements Mo, V, Te, Nb, Pd, and O, respectively; and wherein when a is 1, b is between 0.01 and 1.0, c is between 0 and 1.0, d is between 0 and 1.0, e is between 0 and 0.10, and f is a number to at least satisfy the valence state of the metal elements in the SO catalyst. In an aspect, forming the acetic acid in the SO reactor comprises placing the fresh feed stream in contact with an SO catalyst that includes vanadium.

In an aspect, the method includes producing the acetic acid product stream in one reactor of a number of parallel SO reactors and producing an ethylene product stream in at least one other reactor of the number of parallel SO reactors. In an aspect, an acetic acid product stream from the one reactor of the number of parallel SO reactors is combined with a feed stream provided to the at least one other reactor of the number of parallel SO reactors.

Another embodiment described herein provides a reactor system for producing acetic acid in a selective oxidation process. The reactor system includes a selective oxidation (SO) reactor. The SO reactor includes a number of feed lines including a methane feed line, a carbon dioxide feedline, and a steam feedline. The SO reactor includes an SO catalyst to convert feedstocks, at least in part, to acetic acid, and a reactor effluent line. The reactor system includes a scrubber coupled to the reactor effluent line. The scrubber includes an acetic acid product line and a separated gas outlet. The separated gas outlet is coupled to one of the number of feed lines to recycle at least a portion of a gas stream separated from an acetic acid product stream to the SO reactor. In an aspect, at least two of the number of feed lines are combined to form a single feedline to the SO reactor.

In an aspect, the reactor system includes a plurality of SO reactors in each of the number of SO reactors is selectively coupled to either one of two scrubbers, a first scrubber or a second scrubber. The first scrubber is configured to separate the reactor effluent into the acetic acid product stream in the gas stream and to return the gas stream to an SO reactor coupled to the first scrubber. A second scrubber is configured to separate the reactor effluent into a liquid stream and the gas stream and provide the gas stream to a subsequent SO reactor coupled to the second scrubber. In an aspect, the acetic acid product stream from the first scrubber is coupled to an SO reactor producing ethylene.

In an aspect, the methane feedline is coupled to a natural gas pipeline to provide methane to the SO reactor. In an aspect, the methane feedline is coupled to a fractionator providing ethane. In an aspect, the carbon dioxide feedline is coupled to a carbon dioxide pipeline. In an aspect, the carbon dioxide feedline is coupled to a flue gas line from a combustion process. In an aspect, the reactor system includes a process skid including the SO reactor and the scrubber.

Another embodiment described herein provides a method for producing acetic acid in a selective oxygenation (SO) reactor. The method includes providing a fresh feed stream to the SO reactor. The fresh feed stream includes a methane feed stream provided from a pipeline, a carbon dioxide feed stream provided from a flue gas stream from a combustion process, and a steam feed stream. Acetic acid is formed in the SO reactor, wherein an amount of water added to the SO reactor as the steam feed stream is adjusted to increase selectivity for acetic acid. An acetic acid product stream is separated from a reactor effluent stream in a scrubber. A recycle methane stream is obtained from the scrubber, and at least a portion of the recycle methane stream is combined into the fresh feed stream to the SO reactor.

In an aspect, forming the acetic acid in the SO reactor includes placing the fresh feed stream in contact with an SO catalyst of the formula:

MOₐV_{b}Te_{c}Nb_{d}PdₑO_{f}

wherein a, b, c, d, e, and f are relative atomic amounts of the elements Mo, V, Te, Nb, Pd, and O, respectively; and wherein when a is 1, b is between 0.01 and 1.0, c is between 0 and 1.0, d is between 0 and 1.0, e is between 0 and 0.10, and f is a number to at least satisfy the valence state of the metal elements in the SO catalyst. In an aspect, forming the acetic acid in the SO reactor comprises placing the fresh feed stream in contact with an SO catalyst that includes vanadium.

The details of one or more implementations are set forth in the accompanying drawings and the description below. Other features and advantages will be apparent from the description and drawings, and from the claims.

### BRIEF DESCRIPTION OF DRAWINGS

FIG. 1 is a block diagram of a selective oxidation system for the production of acetic acid from the selective oxidation of light hydrocarbons in the presence of water, in accordance with examples.

FIG. 2 is a simplified process flow diagram of a chemical complex used for the production of ethylene in an oxidative dehydrogenation (ODH) reaction, in accordance with examples.

FIG. 3 is a simplified process flow diagram of an SO reactor system for the production of acetic acid, in accordance with examples.

FIG. 4 is a simplified process flow diagram for using a group of parallel SO reactors wherein one SO reactor is used in a swing capacity to produce acetic acid, while other SO reactors are producing ethylene, in accordance with examples.

FIG. 5 is a simplified process flow diagram of a process skid for the production of acetic acid from methane and carbon dioxide, in accordance with examples.

FIG. 6 is a block flow diagram of a method for the production of acetic acid in a selective oxidation reactor, in accordance with examples.

### DESCRIPTION OF EMBODIMENTS

Other than in the operating examples or where otherwise indicated, all numbers or expressions referring to quantities of ingredients, reaction conditions, etc. used in the specification and claims are to be understood as modified in all instances by the term “about”. Accordingly, unless indicated to the contrary, the numerical parameters set forth in the following specification and attached claims are approximations that can vary depending upon the desired properties, which the present disclosure desires to obtain. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical values, however, inherently contain certain errors necessarily resulting from the standard deviation found in their respective testing measurements.

Also, it should be understood that any numerical range recited herein is intended to include all sub-ranges subsumed therein. For example, a range of “1 to 10” is intended to include all sub-ranges between and including the recited minimum value of 1 and the recited maximum value of 10; that is, having a minimum value equal to or greater than 1 and a maximum value of equal to or less than 10. Because the disclosed numerical ranges are continuous, they include every value between the minimum and maximum values. Unless expressly indicated otherwise, the various numerical ranges specified in this application are approximations.

As used herein, the term “alkane” refers to an acyclic saturated hydrocarbon. In many cases, an alkane consists of hydrogen and carbon atoms arranged in a linear structure in which all of the carbon-carbon bonds are single bonds. Alkanes have the general chemical formula CₙH₂ₙ₊₂. In many embodiments of the disclosure, alkane refers to one or more of methane, ethane, propane, butane, pentane, hexane, octane, decane and dodecane. In particular embodiments, alkane refers to ethane and propane and, in some embodiments, ethane.

As used herein, the term “alkene” refers to unsaturated hydrocarbons that contain at least one carbon-carbon double bond. In many embodiments, alkene refers to alpha olefins. In many embodiments of the disclosure, alkene refers to one or more of ethylene, propylene, 1-butene, pentene, pentadiene, hexene, octene, decene and dodecene. Further, as used herein, the term includes other compounds with carbon-carbon double bonds, such as butadiene, among others. In particular embodiments, alkene refers to ethylene and propylene and, in some embodiments, ethylene.

As used herein, the terms “alpha olefin” or “α-olefin” refer to a family of organic compounds which are alkenes (also known as olefins) with a chemical formula CₓH₂ₓ, distinguished by having a double bond at the primary or alpha (α) position. In many embodiments of the disclosure, alpha olefin refers to one or more of ethylene, propylene, 1-butene, 1-pentene, 1-hexene, 1-octene, 1-decene, and 1-dodecene. In particular embodiments, alpha olefins refer to ethylene and propylene and, in some embodiments, ethylene.

As used herein, the term “essentially free of oxygen” means the amount of oxygen present, if any, remaining in a process stream after the one or more ODH reactors, and in many embodiments after the second reactor as described herein, is low enough that it will not present a flammability or explosive risk to the downstream process streams or equipment.

As used herein, the term “fixed bed reactor” refers to one or more reactors, in series or parallel, often including a cylindrical tube filled with catalyst pellets with reactants flowing through the bed and being converted into products. The catalyst in the reactor may have multiple configurations including, but not limited to, one large bed, several horizontal beds, several parallel packed tubes, and multiple beds in their own shells.

As used herein, the term “fluidized bed reactor” refers to one or more reactors, in series or parallel, often including a fluid (gas or liquid) which is passed through a solid granular catalyst, which can be shaped as tiny spheres, at high enough velocities to suspend the solid and cause it to behave as though it were a fluid.

As used herein, the term “MoVOₓ catalyst” refers to a mixed metal oxide having the empirical formula Mo_{6.5-7.0}V₃O_{d}, where d is a number to at least satisfy the valence of any present metal elements; a mixed metal oxide having the empirical formula Mo_{6.25-7.25}V₃O_{d}, where d is a number to at least satisfy the valence of any present metal elements, or combinations thereof.

As used herein, the term, “selective oxidation” or “SO” refers to an oxidation process that does not proceed to complete thermodynamic oxidation, for example, stopping at products more complex than carbon dioxide and water. As used herein, “oxidative dehydrogenation” or “ODH” is a subset of selective oxidation and refers to processes that couple the endothermic dehydration of an alkane with the strongly exothermic oxidation of hydrogen as is further described herein.

In some embodiments disclosed herein, the degree to which carbon monoxide is produced during an SO process can be mitigated by converting it to carbon dioxide, which can then act as an oxidizing agent. The process can be manipulated so as to control the output of carbon dioxide from the process to a desired level. Using the methods described herein a user may choose to operate in carbon dioxide neutral conditions such that surplus carbon dioxide need not be flared, if contaminated with other materials, or released into the atmosphere, if substantially pure.

Selective oxidation (SO) is generally used in ODH reactions to form ethylene, or other alpha-olefins, from ethane. Examples disclosed herein describe the use of SO to convert carbon dioxide and methane, or other light hydrocarbons, into acetic acid in the presence of steam. An SO catalyst, such as an ODH catalyst, may be used to provide very high selectivity for the formation of acetic acid using conventional ODH operating conditions, for example, at an operating temperature of about 250° C. to about 450° C., a reactor inlet pressure of about 5 psig to about 75 psig, a GHSV of about 200 hr-1 to about 10000 hr-1, a WHSV of about 0.2 hr-1 to about 10 hr-1, and a feed linear velocity of greater than about 5 cm/sec. A stand-alone SO reactor may be used, for example, on a skid-mount. In some examples, an SO reactor in an ODH complex may be used in a swing capacity, allowing other reactors to continue to produce ethylene or other products. The feed can be provided from a steam cracker or other units in an ODH complex. In some cases, the feed can be provided as flue gas from power plants or other combustion processes, providing a path for sequestration of CO₂ emissions.

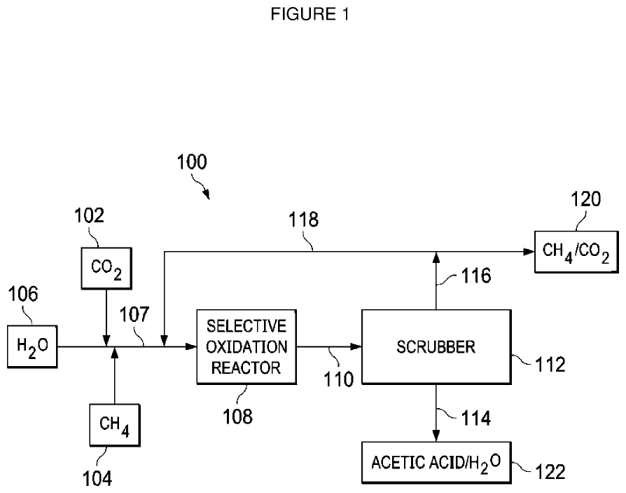

FIG. 1 is a block diagram of a selective oxidation system 100 for the production of acetic acid from the selective oxidation of methane, or other light hydrocarbons, in the presence of water, in accordance with examples. In this example, the feedstocks include carbon dioxide (CO₂) 102, methane (CH₄) 104, and water (H₂O) 106. In some examples, the CH₄ 104 may be replaced with other light hydrocarbons, such as ethane, among others. The feedstocks 102, 104, and 106 are added to a selective oxidation (SO) reactor 108 as a feed stream 107 for conversion of at least a portion of the feedstocks to acetic acid. The feedstocks may be added as individual feed streams, or in combined feed streams with each other and recycled feedstock streams.

The H₂O 106 is generally added as steam to prevent thermal shock from damaging the catalyst bed. The amount of steam added to the SO reactor 108 may be adjusted to control the selectivity. Generally, higher amounts of water added increase the amount of acetic acid formed in the reaction.

In some examples, the CO₂ 102 is provided to the SO reactor 108 as an exhaust stream, or flue gas, from a combustion process. This could be the flue gas from a gas fired turbine, a boiler used for steam generation, or any number of other combustion processes. As water is formed during the combustion process, the flue gas will also provide at least a portion of the water used in the SO reactor 108. Accordingly, the amount of steam used as the H₂O 106 may be adjusted to achieve the desired selectivity. In some examples, CO₂ 102 is provided to the SO reactor 108 from a carbon dioxide pipeline.

The CH₄ 104 may be provided from another hydrocarbon process, such as steam cracking or reforming. In environments in which CH₄ is not synthesized or fractionated from other hydrocarbons, such as a power plant, the CH₄ 104 may be a natural gas feed stream provided from a natural gas pipeline. This may be especially useful in a power plant, as the production of acetic acid may reduce CO₂ emissions, while providing an economically beneficial product for sale. As described herein, the light hydrocarbon is not limited to CH₄. In some examples, other light hydrocarbons, such as ethane, propane, or others, may be used in a light hydrocarbon feed stream to the SO reactor 108. For either the methane or the carbon dioxide, a pipeline may be used to source a portion of the feed stock if insufficient amounts are being provided from a usual source. For example, during a shutdown of a power plant, the CO₂ may be provided from a pipeline to maintain the production of acetic acid, if desired.

Formation of acetic acid from carbon dioxide and methane is speculated to occur based on simplified bulk reaction shown in equation 1.

CH₄+CO₂→C₂H₄O₂ Eqn. 1

The reaction is facilitated by an SO catalyst. In some examples, the SO catalyst has the formula shown in equation 2.

MOₐV_{b}Te_{c}Nb_{d}PdₑO_{f} Eqn. 2

In this formula a, b, c, d, e, and f are relative atomic amounts of the elements Mo, V, Te, Nb, Pd, and O, respectively. When a is 1, b is between 0.01 and 1.0, c is between 0 and 1.0, d is between 0 and 1.0, e is between 0 and 0.10, and f is a number to at least satisfy the valence state of any metal elements present in the SO catalyst. Another example of a catalyst that may be used in processes is shown in the formula of equation 3.

Mo_{6.25-7.25}V₃O_{d} Eqn. 3

In equation 3, d is a number to at least satisfy the valence of the any present metal elements in the catalyst. In other examples, the SO catalyst includes vanadium in addition to any other components, such as mixed metal oxides, that are present.

As used herein, the phrase “to at least satisfy the valence” indicates that additional oxygen may be present. In some embodiments, oxygen is absorbed into the catalyst as lattice oxygen. The lattice oxygen may participate in the catalytic reactions through the transfer of oxygen to the hydrocarbon substrate. This temporarily generates a vacancy, or defect, in the catalyst that is replenished by other oxygen atoms or molecules absorbed in the lattice or external to the catalyst. Accordingly, the catalyst can rely on the ability of the metal oxide to form phases that are not stoichiometric to promote reactions.

In some embodiments, oxygen may be present on the surface of the catalyst in the form of hydroxyl groups. In these embodiments, the amount of oxygen for at least a portion of the metals present is double the amount needed to satisfy the valences of the metals as a hydrogen atom is also present.

Accordingly, there is generally more oxygen in the catalyst than is required to satisfy the valences of the metals. The measurement of the oxygen may be performed by any number of different techniques, such as inductively coupled plasma-mass spectrometry (ICP-MS), among others. The oxygen present beyond that needed to satisfy the valence states is a combination of lattice oxygen and hydroxyl groups, as well as any other oxygen containing groups, such as carbonate groups and the like.

In some examples, the catalyst may be supported on, or agglomerated with, a binder. Some examples of binders include acidic, basic, or neutral binder slurries of TiO₂, ZrO₂, Al₂O₃, AlO(OH) and mixtures thereof. Another useful binder includes Nb₂O₅. The agglomerated catalyst may be extruded in a suitable shape, such as rings, spheres, saddles, cylinders, and the like of a size typically used in fixed bed reactors. When the catalyst is extruded, various extrusion aids known in the art can be used. In some cases, the resulting support may have a cumulative surface area, as measured by BET, of less than about 35 m²/g, less than about 20 m²/g, or less than about 3 m²/g, and a cumulative pore volume from about 0.05 to about 0.50 cm³/g.

The reactor effluent 110 is sent to a scrubber 112, for the removal of acetic acid, other oxygenates, and water. The scrubber 112 may include any number of configurations, such as a heat exchanger followed by a flash vessel or quench tower, which condenses liquids 114 formed in the SO reactor 108 and separates them from gases 116. Increasing the fraction of the recycle feed 118 that is returned to the SO reactor 108 may increase the total conversion of the CO₂ 102 and CH₄ 104 to acetic acid. In examples, the conversion rate of CO₂ 102 and CH₄ 104 in a single pass through the SO reactor 108 is about 15 wt. %, 10 wt. %, 5 wt. %, or lower, depending on the flow rate through the SO reactor 108.

The gases separated from the liquids 114 include unreacted CO₂ and CH₄ which may be returned to the SO reactor as a recycle feed 118 that is combined with one or more of the feedstocks 102, 104, and 106. In some examples, the gases 116 may be provided as a mixed CH₄/CO₂ product 120 to other processes. The liquids 114 include a mixture of acetic acid and H₂O 122, which may be used as a product directly, or further purified to increase the concentration of the acetic acid for sale. In some examples, at least a portion of the water removed during the purification of the acetic acid may be returned to the process feed, for example, being combined with the H₂O 106.

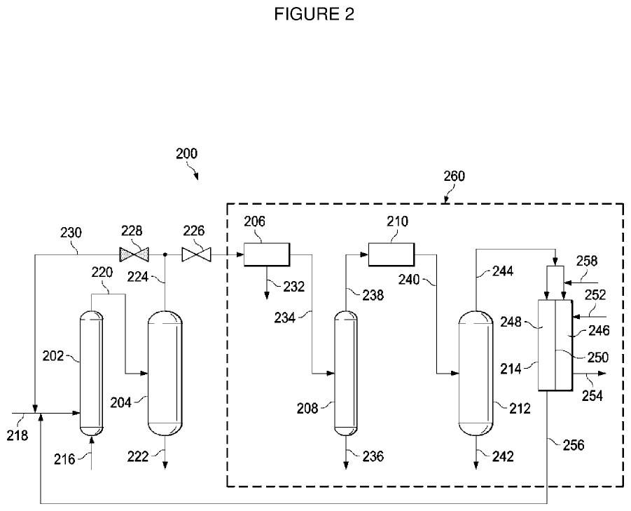

FIG. 2 is a simplified process flow diagram of a chemical complex 200 used for the production of ethylene in an oxidative dehydrogenation (ODH) reaction, in accordance with examples. In this example, the chemical complex 200 includes an SO reactor 202, a scrubber 204, a second reactor 206, an amine wash system 208, a dryer 210, a distillation tower 212, and an oxygen separation module 214. It can be understood that each of these units may include one or more vessels and supporting equipment, such as valves, pumps, sensors, and associated control equipment, such as distributed control systems, and the like.

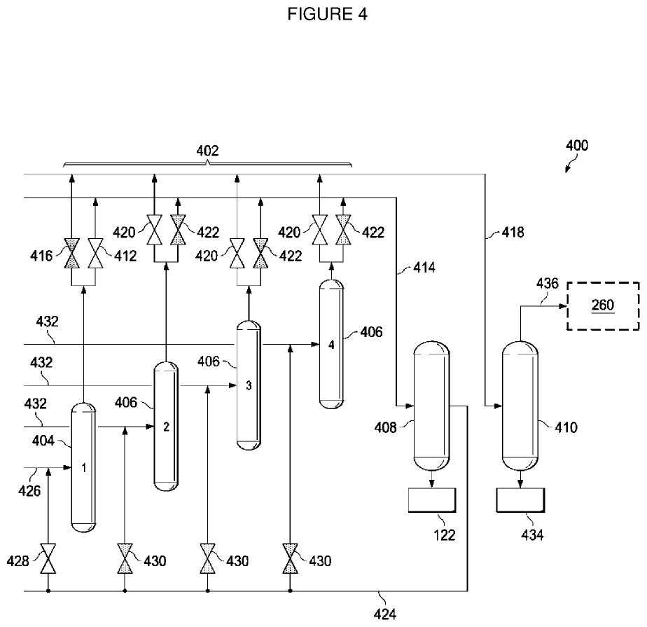

The arrangement of these units is not limited to the arrangement shown. In some examples, the second reactor 206 is not placed directly downstream of the scrubber 204 but is placed further downstream. Depending on the installation environment, not all of the units shown may be present. Further, additional units may be present, for example, a compressor may be placed upstream of the second reactor 206 to increase the pressure of the feed. In some examples, multiple SO reactors are used in a parallel configuration, as described with respect to FIG. 4.

The SO reactor 202 may be a fixed bed reactor or a fluidized bed reactor. The SO reactor 202 includes an SO catalyst capable of catalyzing the oxidative dehydrogenation of alkanes introduced through an alkane line 216. The reaction takes place in the presence of oxygen, which may be introduced through a feed line 218. The feed line 218 may be a single feed line with a combination of the feed stocks or may be divided into any combinations of a light hydrocarbon feed line, a carbon dioxide feed line, and a steam feed line. The SO reaction may also occur in the presence of an inert diluent, such as carbon dioxide, nitrogen, or steam. The inert diluent may be added to the mixture to lower the flammability of the mixture during the SO reaction, or to add additional reactants for the SO reaction used to produce acetic acid, as described herein. It may be noted that the inert diluent is defined with respect to flammability of the reactants. Some of the inert diluents, such as CO₂, may participate in the catalytic reaction in the SO reactor 202. In some examples, all of the reactants are added through the feed line 218. In these examples, upstream equipment may be used to blend the reactants below flammability limits prior to introduction to the SO reactor 202. In some examples, a flooded gas mixer is used to allow mixing of the gases while they are surrounded by a non-flammable liquid, such as water.

The ODH reaction that occurs within the SO reactor 202 may also produce a variety of other products in addition to ethylene and other target olefins. The other products may include carbon dioxide, carbon monoxide, oxygenates, such as acetic acid, and water. These reaction products from the SO reactor 202 are carried by the SO effluent line 220 to the scrubber 204 along with unreacted alkane, the corresponding reacted alkene, residual oxygen, carbon monoxide, and inert diluent. The scrubber 204 quenches the products in the effluent from the SO reactor for the removal of oxygenates and water through the scrubber bottom outlet 222.

The gases that are separated from the reaction products exit the scrubber 204 through the scrubber overhead line 224. The gases may include unconverted alkanes, corresponding alkanes, unreacted oxygen, carbon dioxide, carbon monoxide, and inert diluent. These gases may be directed to the second reactor 206 through an ODH control valve 226 for further processing. As described further herein, an SO recycling valve 228, shown as closed in FIG. 2, is used in other examples to recycle at least a portion of the gases to the fresh feed lines through a recycling line 230.

In this example, the second reactor 206 contains a catalyst with a group 11 metal, for example, with a promoter and support, to react oxygen in the gases with carbon monoxide to form carbon dioxide. The second reactor 206 may be a fixed bed reactor or a fluidized bed reactor. Further, the catalyst can react acetylene with oxygen to reduce or eliminate it. The carbon dioxide from the second reactor 206 can be recycled to the SO reactor 202 through a recycling line 232.

The remaining gases, including a portion of the carbon dioxide, unconverted lower alkanes, the corresponding alkenes, and any other remaining materials are conveyed to the amine wash system 208 through a products line 234. The amine wash system 208 may include an absorber tower in which the gases are contacted with a lean amine, such as diethanolamine, monoethanolamine, or methyldiethanolamine, among others. Any carbon dioxide in the gases is then captured by reaction with the lean amine. After the amine captures the carbon dioxide, it is termed a rich amine. The rich amine is sent to a regenerator in which the carbon dioxide is removed from the rich amine, providing the lean amine that is returned to the absorber tower of the amine wash system 208, and a carbon dioxide stream. The carbon dioxide stream exits the amine wash system 208 through a carbon dioxide outlet 236. In some examples, the carbon dioxide is recycled back to the SO reactor 202 or sold as a product stream. Components of the gases that are not absorbed in the amine wash system 208 exit through an absorber overhead line 238 which conducts the components to the dryer 210.

In various examples, the dryer 210 is a multistage chiller and cryogenic dryer that removes water through a condensation process in a first stage, and then successively removes remaining amounts of water in following stages. In other examples, the dryer 210 is an absorption bed, which absorbs water by flowing the gases through zeolites or other materials. The dried gas is conducted from the dryer 210 through a dry gas line 240 to the distillation tower 212.

The distillation tower 212 may include a single vessel or multiple vessels that perform cryogenic separation. In the distillation tower 212 C₂/C₂₊ hydrocarbons are separated and removed through a distillation bottom outlet 242. The remaining gases include mainly methane, inert diluent, such as nitrogen, and any remaining carbon monoxide. These gases leave the distillation tower through a distillation top outlet 244, and are directed to the oxygen separation module 214.

In this example, the oxygen separation module 214 includes a sealed vessel having a retentate side 246 and a permeate side 248, separated by an oxygen transport membrane 250. As shown, the gases from the distillation top outlet 244 may be directed either to the retentate side 246 or the permeate side 248. In some examples, flow controllers may be included to allow for flow into both sides at varying levels. The oxygen separation module 214 also includes an air input 252 to introduce an oxygen-containing gas into the retentate side 246. Combustion of products in the gases from the distillation top outlet 244 may then heat the oxygen transport membrane 250 to greater than about 850° C., allowing oxygen to pass from the retentate side 246 to the permeate side 248. The oxygen transport membrane 250 blocks other components, besides oxygen, which exit the retentate side 246 of the oxygen separation module 214 through an exhaust 254.

Accordingly, the oxygen separation module 214 provides an oxygen enriched gas from the permeate side 248 that exits the oxygen separation module 214 through an oxygen return line 256. The oxygen return line 256 is then coupled to the feed line 218, or upstream blending equipment, for return to the SO reactor 202. When the gases from the distillation top outlet 244 are directed into the retentate side 246, the concentration of the oxygen in the oxygen return line 256 can approach 99%. When the gases from the distillation top outlet 244 are directed into the permeate side 248, the concentration of the oxygen in the oxygen return line 256 may be about 80% to about 90%, with the remaining gases being carbon dioxide, water, and inert diluent. If the combustion of products in the gases from the distillation top outlet 244 are not sufficient to raise the oxygen transport membrane 250 to this temperature, fuel may be added through a fuel line 258.

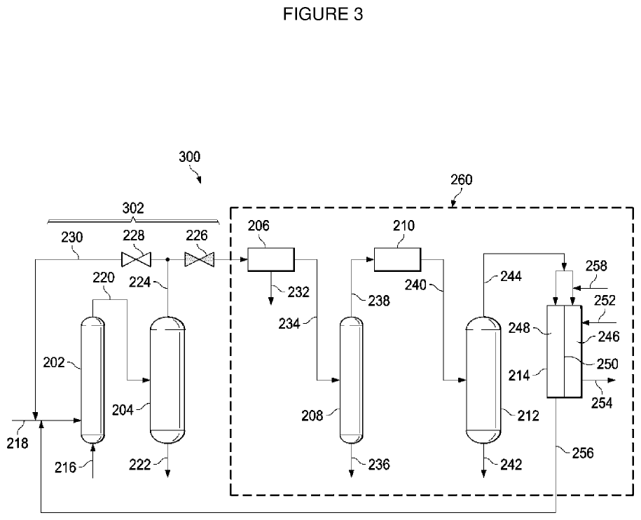

The ODH downstream separation processing taking place in vessels 206, 208, 210, 212, and 214, and their associated equipment, may be grouped into an ODH system 260, as shown in FIG. 2. As described herein, the ODH control valve 226 may direct at least a portion of the gases from the scrubber overhead line 224 to the ODH system 260 for further processing, for example, to sell C₂/C₂₊ from the distillation bottom outlet 242 as a product. The SO recycling valve 228, shown as closed in FIG. 2, is used in other examples to recycle at least a portion of the gases to the fresh feed lines through a recycling line 230, as described further with respect to FIG. 3.

FIG. 3 is a simplified process flow diagram of an SO reactor system 300 for the production of acetic acid, in accordance with examples. Like numbered items are as described with respect to FIG. 2. In this example, the ODH control valve 226 is shown as closed, and the SO recycling valve 228 is shown as open, thus recycling all of the gases from the scrubber overhead line 224 back to the feed line 218. Accordingly, in this example, the reactor 202 and scrubber 204 are functioning as an acetic acid reactor system 302. In the acetic acid reactor system 302 the product is acetic acid produced from the scrubber 204 through the scrubber bottom outlet 222, which is the acetic acid product line.

In some examples, the ODH control valve 226 and the SO recycling valve 228 are proportionally controlled to allow a portion of the gases from the scrubber overhead line 224 to be returned to the SO reactor 202, and another portion of the gases from the scrubber overhead line 224 to be sent on for further processing in the ODH system 260. The acetic acid produced from the scrubber 204 may be processed for sale, as described herein, or may be used to generate further products, such as ethanol or ethylene. In one example, the acetic acid is converted to ethylene by processing it in a second SO reactor system feeding an ODH system. The addition of the acetic acid to the second SO reactor system provides a negative selectivity towards further acetic acid production and an increase in selectivity of ethylene. As used herein, a negative selectivity means that acetic acid is consumed in the process, resulting in increased ethylene production per unit of consumed ethane in the same process.

In various examples, a number of SO reactors are used in a parallel configuration, with at least a portion of the ODH system 260 shared by the SO reactors. In this example, a portion of the SO reactors can be used to produce acetic acid, while other SO reactors may provide feed to the ODH system 260. This configuration is described further with respect to FIG. 4.

FIG. 4 is a simplified process flow diagram 400 for using a group 402 of parallel SO reactors wherein one SO reactor 404 is used in a swing capacity to produce acetic acid, while other SO reactors 406 are producing ethylene, in accordance with examples. Like numbered items are as described with respect to FIGS. 1 and 2.

In this example shown in FIG. 4, the group 402 has four parallel SO reactors that can be fluidically coupled to either one of two downstream scrubbers, an acetic acid scrubber 408, or an ODH scrubber 410. In this example, reactor 1 404 is coupled to the acetic acid scrubber 408 through a valve 412 that is open to an acetic acid header 414. A second valve 416 between reactor 1 404 and an ODH header 418 is closed. The other three reactors 406 in this example, reactor 2, reactor 3, and reactor 4, are coupled to the ODH header 418 through open valves 420, and isolated from the acetic acid header 414 through closed valves 422. Further, a scrubber gas recycle header 424 is coupled to the feed line 426 for reactor 1 404 through a valve 428 that is open to the scrubber gas recycle header 424, while the scrubber gas recycle header 424 is isolated from the remaining reactors 406 by valves 430 that are closed.

Configurations are not limited to the arrangements of the vessels shown. In some examples, a separate ODH scrubber 410 is provided for each of the reactors, and a single acetic acid scrubber 408 is provided to allow one of the reactors to be switched from ethylene production to acetic acid production. In other configurations, multiple vessels are used in place of each of the scrubbers 408 and 410. In these examples, a condenser vessel is used to separate liquid and gas from the reactor effluent. Gas from the condenser is combined with an overhead gas from the liquid and processed in an acetic acid scrubber to remove further traces.

In the example shown, the feed line 426 for reactor 1 may carry a feed that includes methane, carbon dioxide, and steam, providing a high selectivity for the formation of acetic acid in reactor 1. Thus, in this configuration, reactor 1 404 functions as an SO reactor producing acetic acid, as described with respect to the SO reactor 108 of FIG. 1. The feed lines 432 to the remaining reactors 406 carry a feed used for an ODH process, such as ethane, oxygen, carbon dioxide, and an inert diluent.

The acetic acid 122 isolated from the acetic acid scrubber 408 may be sold as a separate product. In some examples, the acetic acid 122 may be combined with the feed to the remaining reactors 406, and added through feed lines 432. As described herein, this may increase the production of ethylene by increasing the selectivity for ethylene over acetic acid in an ODH reaction.

The products from the remaining reactors 406 are carried by the ODH header 418 to the ODH scrubber 410. Oxygenates and water 434 are isolated from the ODH scrubber 410 as described with respect to the scrubber 204 of FIG. 2. A scrubber overhead line 436 from the ODH scrubber 410 feeds the gases to the ODH system 260 for further processing, as described with respect to FIG. 2.

As described herein, the valves 412 and 416 coupling reactor 1 404 to the acetic acid header 414 and the ODH header 418 do not have to be fully open or fully closed. Instead, these valves 412 and 416, and the valve 428, that couples the feed line 426 for reactor 1 404 to the scrubber gas recycle header 424 may be control valves that are partially open or partially closed, as determined by a control system. This may allow a different mixture of products to be formed in the system.

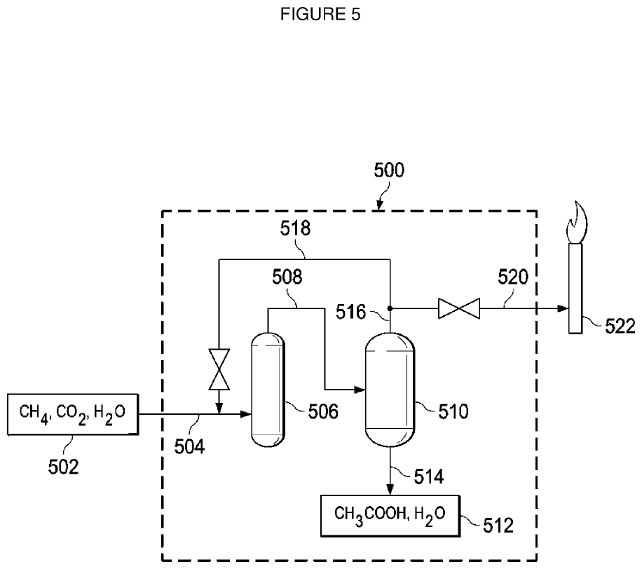

FIG. 5 is a simplified process flow diagram of a process skid 500 for the production of acetic acid from methane and carbon dioxide, in accordance with examples. The process skid 500 allows an acetic acid production reactor to be placed in convenient locations, for example, at power plants sites, refineries, and the like. The feeds 502 to the process skid 500 may include methane, carbon dioxide, and steam. As described herein, the methane may be obtained from a pipeline or other source and the carbon dioxide and steam may be at least partially sourced from a flue gas provided from a combustion process.

The feeds 502 may be combined in the feed line 504, which is provided to the SO reactor 506. As described with respect to the SO reactor 108 of FIG. 1, the SO reactor 506 produces acetic acid at a yield of about 10% with a reactor effluent that includes acetic acid, unreacted methane, unreacted CO₂, and water, among other components. The reactor effluent is carried by an effluent line 508 to a scrubber 510.

As described with respect to the scrubber 112 of FIG. 1, the scrubber 510 quenches the reactor effluent, generating a liquid product 512 that includes acetic acid, water, and any other oxygenated chemicals. The liquid product 512 exits the scrubber 510 through a bottoms line 514, which is the acetic acid product line. Gases isolated from the liquid product 512 exit the scrubber 510 through an overhead line 516. In some examples, the gases are at least partially recycled to the feed line 504 through a recycle line 518. A portion of the gases may be sold or discarded through a gas outlet line 520, for example, being sent to a flare 522, or used in a combustion turbine for power generation or used as a feed effluent diluent for an ODH reactor.

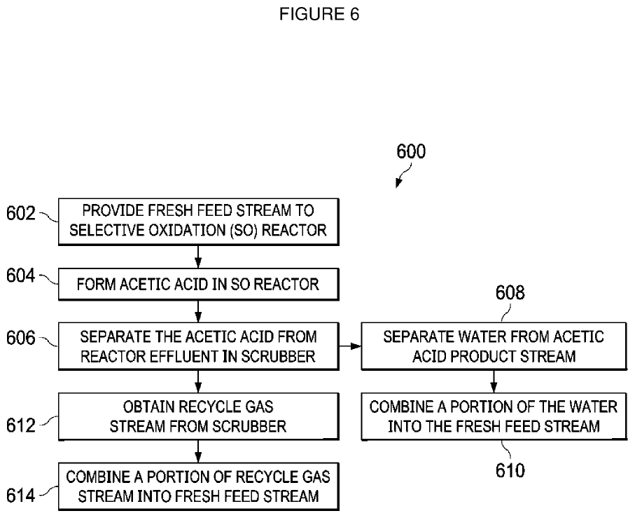

FIG. 6 is a block flow diagram of a method 600 for the production of acetic acid in a selective oxidation reactor, in accordance with examples. The method 600 begins at block 602, when a fresh feed stream is provided to a selective oxidation (SO) reactor. As described herein, the fresh feed stream may include light hydrocarbons, such as methane, carbon dioxide, and water. The SO process described herein is not limited to the use of methane. In some examples, the fresh feed stream may be a mixture of ethane, oxygen, and an inert diluent. In these examples, the reaction may produce acetic acid, ethylene, carbon monoxide, and carbon dioxide. This may be convenient in chemical complexes that already have an ethane feed to the reactors, such as the chemical complex described in the simplified process flow diagrams of FIGS. 2, 3 and 4.

At block 604, acetic acid is formed in the SO reactor, for example, using the catalysts and processes described herein. At block 606, acetic acid is separated from the reactor effluent in a scrubber, as a liquid stream removed from the bottom of the scrubber. At block 608, water is separated from the acetic acid product, for example, to increase the concentration of the acetic acid for sale. At block 610, a portion of the water may be combined into the fresh feed stream provided to the SO reactor.

At block 612, a recycle gas stream is isolated from the scrubber. The recycle gas stream may include unreacted methane and carbon dioxide. At block 614, a portion of the recycle gas stream is combined into the fresh feed stream to the reactor. As described herein, this increases the total yield of the process.

### Examples

The production of acetic acid in a SO reactor was tested using a fixed bed reactor unit (FBRU). The FBRU apparatus comprised two vertically oriented fixed bed tubular reactors in series, each reactor a SS316L tube with an outer diameter of 2.54 cm, an internal diameter of 2.1 cm, a length of 86.4 cm, wrapped in an electrical heating jacket, and sealed with ceramic insulating material. Each reactor contained an identical catalyst bed consisting of extruded pellets, in cylinder form having a diameter of 1.7 mm, lengths ranging from 2 to 10 mm, and comprising a mixture of one weight unit of catalyst to 1.22 units of weight of VERSAL™ Alumina 250 powder. Total weight of the catalyst in each reactor was 171 g catalyst having the formula Mo_{1.0}V_{0.37}Te_{0.23}Nb_{0.14}O_{d=4.97}, with relative atomic amounts of each component to a relative amount of Mo of 1, shown in subscript. The composition was based on ICP-MS measurements where “d” was calculated based on the highest oxide state of the metal elements present.

The temperature of each of the reactors was monitored using corresponding 7-point thermocouples present in each reactor, 5 of which were situated within each catalyst bed, the average of which is reported in Table 1. Temperature control, particularly at lower temperatures, was limited and resulted in fluctuations. Both reactors were being controlled for temperature by controlling the pressure and temperature of a circulating closed loop oil bath which feed into jackets surrounding each reactor. Furthermore, the temperature of the catalyst bed in each reactor was monitored and controlled based on maximum values of the thermocouple measurements using the oil bath. The pressure inside the reactors was controlled and adjusted using a back pressure regulator located downstream of a condenser on the reactor effluent line.

In the FBRU unit, two feed gases can be separately fed through the reactors. One feed gas is the mixture of methane, carbon dioxide, and water, and the other feed gas is air. The first feed gas is used for conducting SO reactions, such as ODH, and the air can be used for catalyst regeneration. The flow of gases is controlled by mass flow controllers. The flow of other materials added, such as water or other liquids, may be controlled by a positive displacement pump.

In addition to the feed gases, an oxygenate-water mixture can be co-fed along with the mentioned feed gases into the inlet of the reactors using a pump. The oxygenate-water mixture evaporates at the inlet of the reactors prior to reaching the catalyst bed, providing additional reactants for testing. The oxygenate may include acetic acid, ethanol, or methanol.

In order to explore the reaction of CO₂/CH₄/H₂O feed mixture on the catalyst, two experiments, A and B, were performed under different inlet reaction pressures. Table 1 shows the operating conditions for the examples, including reaction temperature, the reaction inlet pressure, and the feed composition. The flow rate used, either as gas hourly space velocity (GHSV) or weight hourly space velocity, is also shown, where GHSV is the ratio of gas flow rate under standard conditions for temperature (25° C.) and pressure (100 kilopascals) to the volume of the active phase of the catalyst, and WHSV is the weight of the total feed flowing per unit weight of the catalyst per hour. The results of each experiment including catalyst activity and product distributions are reported in Table 2.

TABLE 1
Reactor Feed Composition Ranges and Operating Condition Ranges
for Experiments

| Experi- | | Reaction | Reaction Inlet | Feed Composition | | |
|---|---|---|---|---|---|---|
| ment | GHSV/WHSV | Temperature | Pressure | (Vol %) | | |
| ID | (h⁻¹) | (° C.) | (psig) | H₂O | CH₄ | CO₂ |
| A | 459/0.53 | 320 | 15-18 | 40 | 30 | 30 |
| B | 459/0.53 | 320 | 51 | 40 | 30 | 30 |

TABLE 2
Catalyst Activity and Product Distribution for Experiments

| | | Reaction | | | Selectivity |
|---|---|---|---|---|---|
| | Reaction | Inlet | CH₄ | CO₂ | (C-atom %) |
| Experiment | Temp. | Pressure | Conversion | Conversion | Normalized |
| ID | (° C.) | (psig) | (C-atom %) | (C-atom %) | Acetic Acid |
| A | 320 | 15-18 | 0.6 | 0.6 | 100 |
| B | 320 | 51 | 0.5 | 0.5 | 100 |

Based on the results in these tables, the following conclusions were made. Acetic acid was the only observed carbon-based product indicating a 100% selectivity towards acetic acid. Increasing the reaction pressure did not increase the CH₄ conversion and CO₂ conversion, implying that the reaction of producing acetic acid from CH₄/CO₂ is likely zero order within the tested pressure interval (15-51 psig). In all cases, the CH₄ conversion and CO₂ conversion were around 1 C-atom %.

A number of implementations have been described. Nevertheless, it will be understood that various modifications may be made without departing from the spirit and scope of the disclosure.

### INDUSTRIAL APPLICABILITY

The present disclosure relates to a method and reactor system for the production of acetic acid by selective oxidation of a feed comprising methane, carbon dioxide and steam.

1. A method for producing acetic acid in a selective oxidation (SO) reactor, comprising:
providing a fresh feed stream to the SO reactor, wherein the fresh feed stream comprises:
a methane feed stream;
a carbon dioxide feed stream; and
a steam feed stream; and
forming acetic acid in the SO reactor,
separating an acetic acid product stream from a reactor effluent stream in a scrubber;
obtaining a recycle gas stream from the scrubber; and
combining at least a portion of the recycle gas stream into the fresh feed stream to the SO reactor.

2. The method of claim 1, wherein adding the fresh feed stream to the SO reactor comprises adding two or more feed streams separately to the SO reactor.

3. The method of claim 1, comprising adjusting an amount of the steam feed stream added to the SO reactor to increase a selectivity for acetic acid.

4. The method of claim 1, comprising using a flue gas stream from a combustion process as the carbon dioxide feed stream.

5. The method of claim 1, comprising using a natural gas stream from a pipeline as the methane feed stream.

6. The method of claim 1, comprising:
separating a recycled water stream from the acetic acid; and
combining at least a portion of the recycled water stream with the steam feed stream.

7. The method of claim 1, wherein forming the acetic acid in the SO reactor comprises placing the fresh feed stream in contact with an SO catalyst of the formula:
MoₐV_{b}Te_{c}Nb_{d}PdₑO_{f}
wherein a, b, c, d, e and f are relative atomic amounts of the elements Mo, V, Te, Nb, Pd, and O, respectively; and wherein when a is 1, b is between 0.01 and 1.0, c is between 0 and 1.0, d is between 0 and 1.0, e is between 0 and 0.10, and f is a number to at least satisfy the valence state of the metal elements in the SO catalyst.

8. The method of claim 1, wherein forming the acetic acid in the SO reactor comprises placing the fresh feed stream in contact with an SO catalyst that comprises vanadium.

9. The method of claim 1, wherein the recycle gas stream comprises methane.

10. The method of claim 1, comprising:
producing the acetic acid product stream in one reactor of a plurality of parallel SO reactors; and
producing an ethylene product stream in at least one other reactor of the plurality of parallel SO reactors.

11. The method of claim 10, comprising combining the acetic acid product stream from the one reactor of the plurality of parallel SO reactors with a feed stream provided to the at least one other reactor of the plurality of parallel SO reactors.

12. A reactor system for producing acetic acid in a selective oxidation process, comprising:
a selective oxidation (SO) reactor, comprising:
a plurality of feed lines comprising:
a methane feed line;
a carbon dioxide feed line; and
a steam feed line;
an SO catalyst to convert feedstocks, at least in part, to acetic acid; and
a reactor effluent line; and
a scrubber coupled to the reactor effluent line, comprising:
an acetic acid product line; and
a separated gas outlet, where the separated gas outlet is coupled to one of the plurality of feed lines to recycle at least a portion of a gas stream separated from an acetic acid product stream to the SO reactor.

13. The reactor system of claim 12, wherein at least two of the plurality of feed lines are combined to form a single feed line to the SO reactor.

14. The reactor system of claim 12, comprising a plurality of SO reactors, wherein each of the plurality of SO reactors is selectively coupled to either one of two scrubbers:
a first scrubber configured to separate the reactor effluent into the acetic acid product stream and the gas stream, and to return the gas stream to an SO reactor coupled to the first scrubber; or
a second scrubber configured to separate the reactor effluent into a liquid stream and the gas stream and provide the gas stream to a subsequent SO reactor coupled to the second scrubber.

15. The reactor system of claim 14, wherein the acetic acid product stream from the first scrubber is coupled to an SO reactor producing ethylene.

16. The reactor system of claim 12, wherein the methane feed line is coupled to a natural gas pipeline to provide methane to the SO reactor.

17. The reactor system of claim 12, wherein the methane feed line is coupled to a fractionator providing ethane.

18. The reactor system of claim 12, wherein the carbon dioxide feed line is coupled to a carbon dioxide pipeline.

19. The reactor system of claim 12, wherein the carbon dioxide feed line is coupled to a flue gas line from a combustion process.

20. The reactor system of claim 12, comprising a process skid comprising the SO reactor and the scrubber.

21. A method for producing acetic acid in a selective oxygenation (SO) reactor, comprising:
providing a fresh feed stream to the SO reactor, wherein the fresh feed stream comprises:
a methane feed stream provided from a pipeline;
a carbon dioxide feed stream provided from a flue gas stream from a combustion process; and
a steam feed stream; and
forming acetic acid in the SO reactor, wherein an amount of water added to the SO reactor as the steam feed stream is adjusted to increase selectivity for acetic acid;
separating an acetic acid product stream from a reactor effluent stream in a scrubber;
obtaining a recycle methane stream from the scrubber; and
combining at least a portion of the recycle methane stream into the fresh feed stream to the SO reactor.

22. The method of claim 21, wherein forming the acetic acid in the SO reactor comprises placing the fresh feed stream in contact with an SO catalyst of the formula:
MoₐV_{b}Te_{c}Nb_{d}PdₑO_{f}
wherein a, b, c, d, e and f are relative atomic amounts of the elements Mo, V, Te, Nb, Pd, and O, respectively; and wherein when a is 1, b is between 0.01 and 1.0, c is between 0 and 1.0, d is between 0 and 1.0, e is between 0 and 0.10, and f is a number to at least satisfy the valence state of the metal elements in the SO catalyst.

23. The method of claim 21, wherein forming the acetic acid in the SO reactor comprises placing the fresh feed stream in contact with an SO catalyst that comprises vanadium.
